(19) United States
(12) Patent Application Publication
(10) Pub. No.: US 2007/0298135 A1
(43) Pub. Date: Dec. 27, 2007

(54) TOPICAL HERBAL FORMULATIONS

(75) Inventor: Lizhen Zhao, Nanjing (CN)

Correspondence Address: SEED INTELLECTUAL PROPERTY LAW GROUP PLLC, 701 FIFTH AVE, SUITE 5400, SEATTLE, WA, 98104, US

(73) Assignee: BIOBOTANIC CORP., East Providence, RI (US)

(21) Appl. No.: 11/750,125

(22) Filed: May 17, 2007

Related U.S. Application Data
(60) Provisional application No. 60/801,971, filed on May 18, 2006.

(51) Int. Cl. A61K 36/896; A61K 36/708; A61P 17/02

(52) U.S. Cl. 424/753

(57) ABSTRACT

An herbal formulation is described as an effective treatment of burn injuries and chronic wounds. The herbal formulation comprises an extract of Paris and Sanguisorba prepared by infusing Paris and Sanguisorba in a liquid medium having a boiling point of no more than 160° C.

### CROSS-REFERENCE TO RELATED APPLICATION

This application claims the benefit under 35 U.S.C. § 119(e) of U.S. Provisional Patent Application No. 60/801,971 filed May 18, 2006, which is incorporated herein by reference in its entirety.

### BACKGROUND

1. Technical Field

The present invention relates to herbal formulations and applications thereof, such as their use to treat injuries to skin, including burns and other acute or chronic wounds.

2. Description of the Related Art

Burns to skin can be caused by thermal, chemical, or electrical contact. Chronic wounds are commonly caused by acute wounds that failed to heal in a timely manner, or skin ulcers caused by systemic problems such as diabetes, bacterial infection or repeated pressure. All injuries to the skin compromise the skin's mechanical integrity, the degree of compromise varying from superficial disruption of the outermost layer of the skin to complete destruction of all layers of skin. Because skin is the body's first defense against environmental microorganisms as well as against fluid loss, a burn that destroys this barrier can cause life-threatening complications for the victim.

At the site of a wound, the victim can be affected by, for example, pain, local infection, wound edema, loss of intravascular fluid volume due to increased vascular permeability, or a combination thereof. Systemic effects include, for example, hypovolemic shock, systemic infection, respiratory tract injury, or a combination thereof. The systemic effects often pose a greater threat to the life of the victim than do the localized effects.

Effective burn and chronic wound treatment must prevent infection as well as prepare the wound site for healing and closure. Current wound bed management includes debridement combined with topical and systemic antimicrobial therapy. Debridement is a procedure to remove the damaged or necrotic tissue at a wound site. Timely debridement not only allows for an accurate assessment of the injury, it also prevents microorganisms from invading and thriving in the necrotic tissue. Removal of the necrotic tissue can furthermore promote new tissue growth.

Debridement can take place according to four primary mechanisms: (1) autolytic debridement, which allows for the body to naturally rid itself of necrotic tissue; (2) enzymatic debridement, which uses enzymes to digest necrotic tissue; (3) mechanical debridement, which removes necrotic tissue through rubbing or hydrotherapy (water); and (4) surgical debridement, which debrides the wound with sharp instruments or lasers.

Despite its numerous potential benefits, debridement is an invasive procedure. The invasive nature of the procedure presents the victim with new risks. For example, mechanical and surgical debridement may cause removal of healthy tissues, bleeding and delayed healing. In particular, debridement may cause fresh trauma to the skin and induce formation of scar tissue. Surgical debridement can also be extremely painful.

Accordingly, there remains a need in the art for an effective and non-invasive regimen for managing injuries to skin caused by burn, acute or chronic wounds.

### BRIEF SUMMARY

According to the present invention, one embodiment provides an herbal formulation based on Chinese medicinal herbs. The herbal components of the formulation synergistically produce pain relieving, anti-swelling, anti-inflammatory, antimicrobial and wound healing promotion effects. In addition, the formulation of the present invention provides a non-invasive solution to necrotic tissue removal. The herbal formulation comprises an extract of Paris Polyphylla Smith (“Paris”) and Sanguisorba prepared by infusing Paris and Sanguisorba in a liquid medium having a boiling point of no more than 160° C.

In another embodiment, a method is described for preparing an herbal formulation, comprising:

a) infusing Paris and Sanguisorba in a liquid medium having a boiling point of no more than 160° C. to form a mixture;

(b) allowing the mixture to separate to a liquid phase and a solid phase; and

(c) separating the liquid phase from the solid phase, wherein the liquid phase comprises active substances from Paris and Sanguisorba.

In another embodiment, a method for treatment of a mammal is described, the method comprising applying to an affected area caused by burn, a skin lesion or an ulcer, an effective amount of an herbal formulation comprising an aqueous extract of Paris and Sanguisorba.

In a further embodiment, a method of treatment of a mammal is described, the method comprising applying to an affected area caused by burn, a skin lesion or an ulcer, an effective amount of an herbal formulation prepared by a) infusing Paris and Sanguisorba in a liquid medium having boiling point of no more than 160° C. to form a mixture; (b) allowing the mixture to separate to a liquid phase and a solid phase; and (c) separating the liquid phase from the solid phase, wherein the liquid phase comprises active substances from Paris and Sanguisorba.

### DETAILED DESCRIPTION

The present invention provides herbal formulations and methods of making and using the same. In particular, the herbal formulations are based on liquid extracts of Chinese medicinal herbs. These medicinal herbs contain active substances that synergistically prevent infection, hemorrhage, edema, and local swelling at a wound site caused by burns or skin ulcers. Furthermore, the herbal formulations cause efficient and non-invasive removal of necrotic tissue at the wound site. The herbal formulations of the present invention are suitable for treating acute burn wounds as well as chronically unhealed burn wounds and other chronic wounds.

A. Herbal Formulations

Typically, therapeutic herbal formulations based on several Chinese medicinal herbs are designed to stimulate and enhance the functionality of each ingredient. At the same time, herbal formulations also aim at suppressing and counteracting toxicity, as well as avoiding incompatibility of each ingredient. Therefore, the balance and interaction of all the ingredients are considered more important than the effect of the individual ingredients. In contrast to a typical Western medication, which focuses on a single chemical entity that targets a single organism or organ, herbal formulations often rely on the combined and synergetic effects of the several herbs to target multiple indications of a disease.

In one embodiment, the present invention provides an herbal formulation comprising an extract of Paris and Sanguisorba prepared by infusing Paris and Sanguisorba in a liquid medium having a boiling point of no more than 160° C.

Paris, also known in Chinese medicinal herbology as Qian Ye Yi Zhi Hua, Zao Xiu or Chong Lou, represents a genus of herbal species that share similar pharmacological actions. The species include, but are not limited to: Paris axialis Li, Paris bashanensis Wang et Tang, Paris croquistii (Takht.) H. Li, Paris dunniana Levl. var oligophylla Wang et Tang, Paris fargesii Franch., Paris fargesii Franch. var. petiolata (Baker ex C. H. Wright) Wang et Tang, Paris forrestii (Takht.) H. Li, Paris incompleta, Paris japonica Franch., Paris lancifolia Hayata, Paris polyphylla Smith, Paris polyphylla Smith var. apetala Hand. Mazz, Paris polyphylla Smith var. appendiculata Hara, Paris polyphylla Smith var. brevipetala Y. K. Yang, Paris polyphylla Smith var. chinensis (Franch.) Hara, Paris polyphylla Smith var. latifolia Wang et Chang, Paris polyphylla Smith var. pseudothibetica H. Li, Paris polyphylla Smith var. pseudothibetica H. Li f. microsepala H. Li, Paris polyphylla Smith var. platypetala Franch., Paris polyphylla Smith var. stenophylla Franch., Paris polyphylla Smith var. thibetica (Franch.) Hara, Paris polyphylla Smith var. wallichii Hara, Paris polyphylla Smith var. yunnanensis (Franch.) Hand. Mazz., Paris pubescens (Hand. Mazz.) Wang et Tang., Paris quadrifolia L., Paris tetraphylla A. Gray, Paris thibetica Franch., Paris verticillata M. Bieb., Paris vietnamensis (Takht.) H. Li, Paris violacea Levl. (P. marmorata Stearn), Paris wenxianensis Z. X. Peng et R. N. Zhao.

The pharmacological actions of Paris include, but are not limited to, anti-tumor, anti-bacterial, anti-inflammatory, pain-relieving, and hemostatic functions. In a certain embodiment, Paris extract in the liquid medium as defined herein contains diosgenin, dioscin and Pariphyllin a, b. It is believed that Diosgenin's water or alcohol solution has an inhibitory effect on certain DNA transcriptase. Dioscin is believed to be an active ingredient for analgesia, sedation, anti-inflammation and hemostasis.

Sanguisorba, known in Chinese medicinal herbology as Di Yu, represents a genus of herbal species that share similar pharmacological actions. The species include, but are not limited to: Sanguisorba alashanica Lioce et Li, Sanguisorba alpine Bunge, Sanguisorba applanta Yu et Li, Sanguisorba applanta Yu et Li var. villosa Ye et Li, Sanguisorba canadensis, Sanguisorba filiformis (Hook. f.) Hand.-Mazz., Sanguisorba hakusanensis, Sanguisorba minor Scop., Sanguisorba officinalis L., Sanguisorba officinalis L. var. carnea (Fisch.) Regel ex Maxim., Sanguisorba officinalis L. var. glandulosa (Kom.) Worosch., Sanguisorba officinalis L. var. latifolia Liou et C. Y. Li, Sanguisorba officinalis L. var. longifolia (Bert.), Sanguisorba sitchensis C. A. Mey., Sanguisorba tenuifolia Fisch. Ex Link, Sanguisorba tenuifolia Fisch. ex Link var. alba Trautv. Et Meyer.

The pharmacological actions of Sanguisorba include, but are not limited to hemostatic, anti-bacteria, anti-inflammatory and pain relieving. In a certain embodiment, Sanguisorba's extract in the liquid medium as defined herein contains Ziyu-glycoside I, II; Sanguiborbin A, B, E, Saponins, Triterpenoidal Saponin, and Tannin substances.

“Herb” refers to a plant's matter, including its flower, barks, leaves, fruit, green stems, rhizome and roots, that contains one or more pharmacologically active substances.

“Extraction” refers to a process in which one or more substances having pharmacological activities, or “active substances”, are isolated from an herb or a mixture of herbs. Typically, the active substances in the herb(s) preferentially dissolve into a liquid medium. The liquid medium containing the active substances extracted from one or more herbs, also referred to “liquid phase”, can thereafter be separated from the herb(s) to provide an “herbal extract” or “extract”.

“Infusion” refers to a process to carry out the extraction, in which the herbs are soaked in a liquid medium to enable the active substances to enter the liquid medium. Typically, the herbs are allowed to infuse for a sufficient amount of time for the active substances in the liquid phase to reach an equilibrium, such that no more active substances can be further extracted even with prolonged infusion.

The nature and the yield of an active substance extracted are dependent upon its solubility in a given liquid medium. A liquid medium may be polar or non-polar. Examples of the polar liquid medium include water, alcohols, acetone, or a mixture thereof. Examples of the non-polar liquid medium (or lipophilic solvent) include an oil, such a plant-based oil, an animal-based oil or a mineral oil. In certain embodiments, an active substance may be preferentially soluble in one type of medium over another. For example, Doscin, an active substance in Paris, is soluble in methanol, ethanol, n-butanol, water, hydrate methanol and ethanol, but insoluble in lipophilic organic solvent. An active substance is “soluble” in a liquid medium if its solubility in the liquid medium is at least 0.1 mg/ml (w/v) at room temperature (about 25° C.).

A liquid medium may be volatile, having a boiling point of no more than 160° C. Examples of volatile liquid media include water, an alcohol, acetone or a mixture thereof. Examples of the alcohol include methanol, ethanol, isopropanol, butanol, and combinations thereof.

“Extracted substances” refers to one or more active substances isolated from the herbs. In certain embodiments, the extracted substances are in a concentrated form by removing the liquid medium. For example, the extract of Paris and Sanguisorba can be concentrated by allowing the liquid medium to evaporate. Once the liquid medium is removed, the concentrated extract may be in the form of a liquid, for example, an oily substance. It may also take the form of a semi-solid or solid. The concentrated extract can be further formulated into various forms by combining with a carrier material, such as a cream, an oil or a gel. In other embodiments, the extract can be used directly or further formulated without the removal of the liquid medium.

“Aqueous extract” refers to an extract of active substances extracted from an herb or a mixture of herbs by infusing the herbs in water.

As noted herein, the biological activity of the herbal formulation is the combined result of a mixture of the active substances. Without wishing to be bound by any particular theory, it is believed that the extract of Paris and Sanguisorba comprises active substances that separate necrotic tissue from healthy tissue at the burn site. The active substances may be of a chemical or a biological nature. “Necrotic tissue” refers to damaged and unviable tissue in any layer of skin caused by a burn, an infection or complications ensuing thereafter. Necrotic tissue tends to adhere to the burn site and provides a breeding ground for bacterial growth. As noted herein, removal of the necrotic tissue (debridement) is critical in preventing infection, control inflammation and promoting tissue regeneration.

In addition, the extract of Paris and Sanguisorba comprises active substances having pain-relieving, hemostatic, broad-spectrum antimicrobial, anti-virus and anti-inflammatory activities. Testing has shown that Paris contains a variety of glycosides having hemostatic ability, and saponins having pain-relieving and anti-inflammatory functions. In a certain embodiment, Sanguisorba extract in the liquid medium as defined herein contains saponins and other glycosides, as well as a variety of tannin compounds. The combined effects of the extract of Paris and Sanguisorba therefore target multiple indications of a typical burn injury, such as local infection, hemorrhage and edema.

In another embodiment, the herbal formulation further comprises an additional antimicrobial herbal extract. Examples of the antimicrobial herbal extract include, but are not limited to, an extract prepared by infusing Rheum, Reynoutria, Coptis, Scutellaria, Phellodendron or any combination thereof in a liquid medium having a boiling point of no more than 160° C.

Rheum, known in Chinese medicinal herbology as Da Huang, represents a genus of herbal species that share similar medicinal functions. The species include, but are not limited to: Rheum acuminatum Hook. f. et Thoms, Rheum alexandrae Batalin, Rheum altaicum A, Los., Rheum collinianum Baillon, Rheum compactum L., Rheum coreanum Nakai, Rheum delavyi Franch., Rheum diawoo Makino, Rheum emodi Wall., Rheum forrestii Diels, Rheum franzenbachii Munt., Rheum glabricaule G. Sam, Rheum glabricaule G. Sam. f. brevilobatum G. Sam., Rheum globulosum Gage, Rheum hotaoense C. Y. Cheng et T. C. Kao, Rheum ihasaense, Rheum inopinatum Prain, Rheum kialense Franch., Rheum likiangense Sam., Rheum liupanshanense Cheng et Kao, Rheum maritimus L., Rheum moorcroftianum Royle, Rheum nanum Siev. ex Pall., Rheum nobile Hook. f. et Thoms., Rheum officinale Baill., Rheum palmatum L., Rheum pumilum Maxim, Rheum qinlingenese Y. K. Yang, D. K. Zhang et J. K. Wu, Rheum racemiferum Maxim., Rheum reticulatum A. Los., Rheum rhaponticum L., Rheum rhizostachyum Schrenk, Rheum rhomboideum, Rheum ribes L., Rheum rupestre Litv., Rheum scaberrimum Lingelsh., Rheum spiciforme Royle, Rheum sublanceolatum C. Y. Cheng et Kao, Rheum tanguticum Maxim. ex Regel, Rheum tanguticum Maxim. ex Regel var. liupanshanense C. Y. Cheng et T. C. Kao, Rheum tibeticum Maxim. ex Hook. f., Rheum undulatum L., Rheum uninerve Maxim., Rheum webbianum Royle, Rheum wittrockii Lundstr.

In certain embodiments, chemical substances such as Emodin and Chrysophanic acid in Rheum are extracted by the liquid medium, as defined herein.

Reynoutria, known in Chinese medicinal herbology as Hu Zhang, represents a genus of herbal species that share similar medicinal functions. The species include, but are not limited to: Polygonum cuspidatum Sieb. et Zucc., Reynoutria japonic Houtt.

In one embodiment, chemical substances such as Emodin, trans-resveratrol and Tannin in Reynoutria are extracted in the liquid medium, as defined herein.

Coptis, known in Chinese medicinal herbology as Huang Lian, represents a genus of herbal species that share similar medicinal functions. The species include, but are not limited to: Coptis anemonaefolia S. et Z., Coptis brachypetala S. et z., coptis brachypetala s. et. z. var. pygmaca Miq., Coptis chinensis Franch, Coptis chinensis Franch var. brevisepala W. T. Wang et Hsiao, Coptis deltoidea C. Y. Cheng et Hsiao, Coptis gulinensis, Coptis japonica Makino, Coptis japonica Makino var. major Satake, Coptis japonica makino var. dissecta Nakai, Coptis omeiensis (Chen) C. Y. Cheng, Coptis omeiensis (Chen) C. Y. Cheng var. stolonifera S. L. Zhang, Coptis quinquefolia Miq., Coptis quinquesecta W. T. Wang, Coptis teeta Wall., Coptis trifolia Salisb.

In one embodiment, chemical substances such as Berberine and Coptisine in Coptis are extracted in the liquid medium, as defined herein.

Scutellaria, known in Chinese medicinal herbology as Huang Qin, represents a genus of herbal species that share similar medicinal functions. The species include, but are not limited to: Scutellaria alaschanica Tschern., Scutellaria alpina L., Scutellaria altissima L., Scutellaria amoena C. H. Wright, Scutellaria amoena C. H. Wright var. cineria Hand.-Mazz., Scutellaria baicalensis Georgi, Scutellaria bambusetorum C. Y. Wu, Scutellaria bartata D. Don, Scutellaria canescens, Scutellaria caryopteriodes Hand.-Mazz., Scutellaria caudifolia Sun ex C. H. Chow var. obliguifolia C. Y. Wu et S. Chow, Scutellaria coleifolia Levl. (S. violacea var. sikkimensis Hook. f.), Scutellaria columnae, Scutellaria cordifolia, Scutellaria dependens Maxim., Scutellaria discolor Wall. ex Benth., Scutellaria discolor Wall. ex Benth. var. hirta Hand.-Mazz., Scutellaria epilobiifolia, Scutellaria forrestii Diels, Scutellaria franchetiana Levl., Scutellaria galericulata L., Scutellaria hypericifolia Levl., Scutellaria incana, Scutellaria indica L., Scutellaria indica L. var. elliptica Sun ex C. H. Hu, Scutellaria indica L. var. humilis Mak., Scutellaria indica L. var. parvifolia (Makino) Mikino, Scutellaria indica L. var. subacaulis (Sun ex C. H. Hu) C. Y. Wu et C. Chen, Scutellaria integrifolia, Scutellaria lateriflora L., Scutellaria likiangensis Diels, Scutellaria macrantha Fisch., Scutellaria moniliorrhiza Kom., Scutellaria obtusifolia Hemsl., Scutellaria obtusifolia Hemsl. var. trinervata (Vant.) C. Y. Wu et H. W. Li, Scutellaria omeiensis C. Y. Wu, Scutellaria omeiensis C. Y, Wu var. serratifolia C. Y. Wu et S. Chow, Scutellaria orthocalyx Hand.-Mazz., Scutellaria pekinensis Maxim., Scutellaria pekinensis Maxim. var. purpureicaulis (Migo) C. Y. Wu et H. W. Li, Scutellaria pekinensis Maxim. var. ussuriensis (Regel) Hand.-Mazz., Scutellaria peregrina, Scutellaria pingbienensis C. Y. Wu et H. W. Li, Secutellara prostrata Jacq., Scetellaria purpureocardia C. Y. Wu, Scutellaria quadrilobulata Sun ex C. H. Hu, Scutellaria regeliana Nakai, Scutellaria regeliana Nakai var. ikonnikovii (Juz.) C. Y. Wu et H. W. Li, Scutellaria rehderiana Diels, Scutellaria salviifolia Bentham, Scutellaria scandens D. Don, Scutellaria scordifolia Fisch. ex Schrank, Scutellaria scordifolia Fisch. ex Schrank var. villosissima C. Y. Wu et W. T. Wang, Scutellaria sessilifolia Hemsl., Scutellaria shweliensis W. W. Smith, Scutellaria strigilosa Hemsl., Scutellaria tayloriana Dunn, Scutellaria tenax W. W. Smith, Scutellaria tenax W. W. Smith var. patentipilosa (Hand.-Mazz.) C. Y. Wu, Scutellaria tournefortii Benth., Scutellaria tuberifera C. Y. Wuet C. Chen, Scutellaria violacea var. sikkimensis Hook. f., Scutellaria viscidual Bunge, Scutellaria yunnanensis Levl., Scutellaria yunnanensis Levl. var salicifolia Sun ex C. H. Hu.

In certain embodiments, chemical substances such as baicalin, Wogonoside and beta-sitosterol in Scutellaria are extracted in the liquid medium, as defined herein.

Phellodendron, known in Chinese medicinal herbology as Huang Bai, represents a genus of herbal species that share similar medicinal functions. The species include, but are not limited to: Phellodendron amurense Rupr. f. molle (Nakai) Y. C. Chu (Ph. molle Nakai), Phellodendron amurense Rupr.var. japonicum Ohwi, Phellodendron chinense Schneid., Phellodendron chinense Schneid. var. falcatum Huang, Phellodendron chinense Schneid. var. glabriusculum Schneid. (Ph. chinense, Schneid. var. falcatum Huang), Phellodendron insulare Nakai, Phellodendron japonicum Nakai, Phellodendron lavalii, Phellodendron molle Nakai, Phellodendron sachalinensis Sarg., Phellodendron sinii Y. C. Qu, Phellodendron wilsonii Hayata et Kanehira.

In certain embodiments, chemical ingredients such as Berberine, beta-sitosterol, and phellodendrine in Phellodendron are extracted in the liquid medium, as defined herein.

It is believed that the extract of the above anti-infective herbs target organisms such as Aspergillus, Mucor, Enteroacter cloacae, Aeromonas hydrophila and Enterococcus faecalis bacteria, which have been found to frequently cause infections at a burn wound.

In another embodiment, the herbal formulation further comprises an anti-inflammatory herbal extract. Examples of the anti-inflammatory herbal extract include, but are not limited to, an extract prepared by infusing Dendranthema, Ligustrum, Ilex or a combination thereof in a liquid medium having a boiling point of no more than 160° C.

Ilex, also known in Chinese medicinal herbology as Dong Qing, represents a genus of herbal species that share similar medicinal functions. The species include, but are not limited to: Ilex bioritsensis Hayata, Ilex angulata Merr. Et Chun, Ilex chinensis Sims, Ilex purpurea Hassk., Ilex cornuta Lindl. Et Paxt.

Dendranthema, also known in Chinese medicinal herbology as Ye Ju Hua., represents a genus of herbal species that share similar medicinal functions. The species include, but are not limited to: Dendranthema chanetii (Levl.) Shih, Dendranthema indicum (L.) Des Moul. (Chrysanthemum indicum L.), Dendranthema lavandulifolium (Fisch. ex Trautv.) Ling et Shih, Dendranthema lavandulifolium (Fisch. ex Trautv.) Ling et Shih var. seticuspe (Maxim.) Shih, Dendranthema lavandulifolium (Fisch. ex Trautv.) Ling et Shih var. tomentellum (Hand.-Mazz.) Ling et Shih, Dendranthema mongolicum (Ling) Tzvel., Dendranthema morifolium (Ramat.) Tzvel. (chrysanthemum morifolium Ramat.), Dendranthema naktongense (Nakai) Tzvel., Dendranthema potentilloides (Hand.-Mazz.) Shih, Dendranthema vestitum (Hemsl.) Ling, Dendranthema zawadskii (Herb.) Tzvel.

Ligustrum, known in Chinese medicinal herbology as Si Ji Qing, represents a genus of herbal species that share similar medicinal functions. The species include, but are not limited to: Ligustrum lucidum Ait., Ligustrum malongense B. S. Sun, Ligustrum molliculum Hance (L. acutissimum Koehne), Ligustrum obtusifolium Sieb. et Zucc., Ligustrum japonicum Thumb., Ligustrum japonicum Thumb. var. rotundifolium Nichols, Ligustrum henryi Hemsl., Ligustrum henryi Hemsl. var. longitubum P. S. Hsu, Ligustrum sinense Lour., Ligustrum sinense Lour. var. coryanum (W. W. Smith) Hand.-Mazz. (L., coryanum, W. W. Smith).

Additional herbs containing anti-inflammatory active substances include Patrinia, and Lithospermum (or Arnebia). Patrinia, known in Chinese medicinal herbology as Bai Jiang Cao, represents a genus of herbal species that share similar medicinal functions. The species include, but are not limited to: Patrinia scabiosaefolia Fisch ex Link, Patrinia villosa. Juss., Patrinia villosa. (Thumb.) Juss. ssp. punctifolia H. J. Wang. Lithosperumum (or Arnebia) represents two genuses of herbal species that share similar medicinal functions. The species include, but are not limited to: Lithospermum erythrorhizon Sieb. et. Zucc., Arnebia euchroma (Royle) Johust, Arnebia guttata Bunge.

In another embodiment, the herbal formulation further comprises a hemostatic herbal extract. Examples of the hemostatic herbal extract include, but are not limited to, an extract prepared by infusing Panax, Bletilla, Rheum or a combination thereof in a liquid medium having a boiling point of no more than 160° C.

Panax, also known in Chinese medicinal herbology as Ren Shen, San Qi, represents a genus of herbal species that share similar medicinal functions. The species include, but are not limited to: Panax ginseng C. A. Meyer; Panax pseudo-ginseng Wall. Panax pseudo-ginseng Wall. Var. japonicus (C. A. Meyer) Hoo et Tseng; Panax pseudo-ginseng Wall. Var. notoginseng (Burkill) Hoo et Tseng; Panx quinquefolium L.; Panx stipuleanatus H. J. Tsai et K. M. Feng; Panax trifolium L.; Panax zingiberensis C. Y. Wu et K. M. Feng.

Bletilla, also known in Chinese medicinal herbology as Bai Ji., represents a genus of herbal species that share similar medicinal functions. The species include, but are not limited to: Bletilla formosana (Hayata) Schletcht.; Bletilla formosansa Schlecht. form a kotoensis T P. Lin; Bletilla formosansa Schletcht var. limprichtii Schletcht.; Bletilla ochracea Schltr.; Bletilla sinensis (Rolle) Schlecht.; Bletilla striata (Thunb. Et Murray) Reichb. F.; Bletilla striata (Thunb. Et Murray) Reichb. F. var. gebina (Lindl.) Reichb.f.

The extract of Paris and Sanguisorba can be prepared by infusing Paris and Sanguisorba in a liquid medium such as water, an alcohol or a mixture thereof.

Paris and Sanguisorba can be grounded up prior to infusion in order to increase the efficiency of the extraction. Heating and agitation can also be employed to assist the extraction. The infusion temperature is typically kept below the boiling point of the solvent. For example, the mixture of Paris and Sanguisorba in water can be infused at temperatures up to 100° C. Typically, the infusion temperature can be in the range of 20° C. to 85° C. More typically, the infusion temperature can be in the range of 25° C. to 65° C.

Advantageously, the infusion and extraction are carried out under mild conditions without subjecting the herbs to high temperatures. Unlike some traditional Chinese herbal concoctions, in which herbs are frequently fried in vegetable oils at temperature higher than 200° C., the herbal extract of the present invention prevents the active substances from evaporating or decomposing.

The infusion time may differ depending on the infusion temperature. Typically, the lower the temperature, the longer the infusion time needed. For example, at 5-10° C., the infusion may take 12-20 weeks. At 30-45° C., the infusion may take 6-12 weeks; At 60-80° C., the infusion may take 5-15 hours.

During the infusion process, additional herbs having antimicrobial functions can be combined with Paris and Sanguisorba. For example, Rheum, Reynoutria, Coptis, Scutellaria, Phellodendron or a combination thereof can be used to achieve an optimal level of anti-infectiveness. Similarly, additional herbs having anti-inflammatory functions, such as Dendranthema, Ligustrum, Ilex, Patrinia, and Lithospermum (or Arnebia), can also be combined. Similarly, additional herbs having hemostatic functions, such as Panax, Rheum and Bletilla, can also be combined. The extract of all the herbs may be concentrated and further formulated.

Table 1 shows examples of infusion processes useful in preparing the herbal extract described herein.

TABLE 1

| Ingredients (g) | Process 1 | Process 2 | Process 3 | Process 4 |
|---|---|---|---|---|
| Paris | 100 | 100 | 100 | 100 |
| Sanguisorba | 100 | 100 | 100 | 100 |
| Rheum | — | 50 | 100 | 100 |
| Reynoutria | — | — | 50 | 50 |
| Coptis | — | 50 | 50 | 50 |
| Scutellaria | — | — | 50 | 50 |
| Phellodendron | — | — | 50 | — |
| Dendranthema | — | — | 50 | — |
| Ligustrum | 50 | — | — | — |
| liquid medium | 800 | 1200 | 2100 | 2000 |
| temperature (° C.) | 60 | 60 | 25 | 45 |
| time of infusion (days) | 10 hours | 10 hours | 16 weeks | 10 weeks |

In other embodiments, the present invention further provides herbal formulations including the herbal extract combined with a carrier. The carrier is suitable for topical applications of the active substances contained in the extract. Examples of the carriers are typically in the forms of: solutions, ointments, creams, sprays, gels, and lotions.

In particular, in one embodiment, the present invention provides an herbal formulation comprising (1) an extract of Paris and Sanguisorba prepared by infusing Paris and Sanguisorba in a liquid medium having a boiling point no more than 160° C. and (2) water.

Such an aqueous formulation can be directly obtained by extracting Paris and Sanguisorba using water. Alternatively, an extract of Paris and Sanguisorba using a liquid medium other than water can be concentrated followed by mixing with water. The resulting formulation is an aqueous solution, which formulation is also referred to as “aqueous-based formulation”.

In another embodiment, the above aqueous formulation may further comprise an additional antimicrobial herbal extract by infusing Rheum, Reynoutria, Coptis, Scutellaria, Phellodendron or a combination thereof in the liquid medium.

In another embodiment, the above aqueous formulation may further comprise an additional anti-inflammatory herbal extract by infusing Dendranthema, Ligustrum, Ilex, Patrinia, Lithospermum (or Arnebia) or a combination thereof in the liquid medium.

In another embodiment, the above aqueous formulation may further comprise an additional anti-hemorrhage herbal extract by infusing Panax, Bletilla, or a combination thereof in the liquid medium.

Advantageously, an aqueous based formulation provides a moist environment on the wound surface, which benefits migration of epithelial cells during the epithelialization process of wound healing. In a further embodiment, the present invention provides an herbal formulation comprising (1) an extract of Paris and Sanguisorba prepared by infusing Paris and Sanguisorba in a liquid medium having a boiling point no more than 160° C. and removing the liquid medium and (2) an oil.

According to this embodiment, the extract of Paris and Sanguisorba is in a concentrated form following the removal of the liquid medium. The formulation is then prepared by mixing an oil with the concentrated extract Suitable oils include vegetable oil, animal fat or mineral oil. Examples of vegetable oil include, but are not limited to: almond oil, borage oil, sesame oil, canola oil, grape seed oil, jojoba oil, olive oil, soybean oil, sunflower oil and wheat germ oil. The resulting formulation is an ointment.

In another embodiment, the above oil-based formulation may further comprise an additional antimicrobial herbal extract by infusing Rheum, Reynoutria, Coptis, Scutellaria, Phellodendron or a combination thereof in the liquid medium.

In another embodiment, the above oil-based formulation may further comprise an additional anti-inflammatory herbal extract by infusing Dendranthema, Ligustrum, Ilex, Patrinia, and Lithospermum (or Arnebia) or a combination thereof in the liquid medium.

In another embodiment, the above oil based formulation may further comprise an additional hemostatic herbal extract by infusing Panax, Bletilla, Rheum or a combination thereof in the liquid medium.

In a further embodiment, the present invention provides an herbal formulation comprising (1) an extract of Paris and Sanguisorba prepared by infusing Paris and Sanguisorba in a liquid medium having a boiling point no more than 160° C. and (2) a cream.

Cream refers to an oil-in-water or water-in-oil suspension. For example, the extract of Paris and Sanguisorba can be in a concentrated form and mixed with a cream base. Cream bases suitable for topical applications are known to one skilled in the art. Cream bases can provide a moist environment for wound healing without bandage. A cream can be better absorbed by minor damaged skin and healthy skin surrounding the wound area. Alternatively, oil can be added to an aqueous solution of the herbal extract. Suitable oils include those described herein. Optionally, an emulsifier can be added to stabilize the cream. Examples of the emulsifier include but are not limited to lecithin, stearic acid, and lauryl sulfate. The cream formulation is typically a semi-solid at room temperature and becomes liquefied when applied to the wound site. Alternatively, the cream can be a liquid, i.e., a lotion, at room temperature.

In another embodiment, the above cream-based formulation may further comprise an additional antimicrobial herbal extract by infusing Rheum, Reynoutria, Coptis, Scutellaria, Phellodendron or a combination thereof in the liquid medium.

In another embodiment, the above cream-based formulation may further comprise an additional anti-inflammatory herbal extract by infusing Dendranthema, Ligustrum, Ilex, Patrinia, and Lithospermum (or Arnebia) or a combination thereof in the liquid medium.

In another embodiment, the above cream based formulation may further comprise an additional hemostatic herbal extract by infusing Panax, Bletilla, Rheum or a combination thereof in the liquid medium.

In another embodiment, the present invention provides an herbal formulation comprising (1) an extract of Paris and Sanguisorba by infusing Paris and Sanguisorba in a liquid medium having a boiling point no more than 160° C. and (2) a gel base. For example, the extract of Paris and Sanguisorba can be in a concentrated form by removing the liquid medium. Gel refers to a water-soluble semi-solid. Examples of the gel base include Carbomer, Xanthan Gum, Carrageenan, Pluronic, Acacia Gum, Guar Gum, Agar-Agar, alginates, carboxymethyl cellulose, hydroxyethyl cellulose and mixtures thereof.

In another embodiment, the above gel-based formulation may further comprise an additional antimicrobial herbal extract by infusing Rheum, Reynoutria, Coptis, Scutellaria, Phellodendron or a combination thereof in the liquid medium.

In another embodiment, the above gel-based formulation may further comprise an additional anti-inflammatory herbal extract by infusing Dendranthema Ligustrum, Ilex, Patrinia, and Lithospermum (or Arnebia) or a combination thereof in the liquid medium.

In another embodiment, the above gel based formulation may further comprise an additional hemostatic herbal extract by infusing Panax, Bletilla, Rheum or a combination thereof in the liquid medium.

A preferred formulation is aqueous based and comprises the following herbal ingredients (Table 2), infusing at 40° C. for 14 weeks.

TABLE 2

| Latin Names | Part of Herb Used | Ratio (weight) |
|---|---|---|
| Paris Polyphylla Smith | Rhizoma | 2 |
| Sanguisorba officinalis L. | Radix | 2 |
| Rheum offcinale Baill. | Radix et Rhizoma | 2 |
| Scutellaria baicalensis Georgi | Radix | 1 |
| Polygonum cuspidatum Sieb. et Zucc. | Rhizoma | 1 |
| Phellodendron amurense Rupr | Cortex. | 1 |
| Coptis chinensis Franch. | Rhizoma | 1 |
| Dendranthema morifolium (Ramat.) | Flos | 1 |
| Tzvel. | | |
| Purified Water | — | 40 |

Another preferred formulation comprises the following herbal ingredients (Table 3), infusing at 80° C. for 24 hours.

TABLE 3

| Latin Names | Part of Herb Used | Ratio (weight) |
|---|---|---|
| Paris Polyphylla Smith | Rhizoma | 2 |
| Sanguisorba officinalis L. | Radix | 2 |
| Rheum offcinale Baill. | Radix et Rhizoma | 1 |
| Coptis chinensis Franch. | Rhizoma | 1 |
| Purified Water | — | 24 |

Another preferred formulation comprises the following herbal ingredients (Table 4), infusing at 80° C. for 24 hours.

TABLE 4

| Latin Names | Part of Herb Used | Ratio (weight) |
|---|---|---|
| Paris Polyphylla Smith | Rhizoma | 2 |
| Sanguisorba officinalis L. | Radix | 2 |
| Rheum offcinale Baill. | Radix et Rhizoma | 2 |
| Scutellaria baicalensis Georgi | Radix | 1 |
| Polygonum cuspidatum Sieb. et Zucc. | Rhizoma | 1 |
| Coptis chinensis Franch. | Rhizoma | 1 |
| Purified Water | — | 40 |

In other embodiments, the present invention provides a method of making an herbal formulation comprising: (a) infusing Paris and Sanguisorba in a liquid medium having boiling point of no more than 160° C. to form a mixture; (b) allowing the mixture to separate into a liquid phase and a solid phase; and (c) separating the liquid phase from the solid phase, wherein the liquid phase comprises active substances from Paris and Sanguisorba.

In one embodiment, the liquid medium is water. In another embodiment, the liquid medium is an alcohol. In another embodiment, the liquid medium is a mixture of water and an alcohol. In yet another embodiment, the liquid medium is acetone.

In another embodiment, the method further comprises: after the separating step, infusing the solid phase with fresh liquid medium, and repeating steps (b) and (c).

In another embodiment, the method further comprises: after the separating step, removing the liquid medium in the liquid phase.

In another embodiment, the method further comprises mixing an antimicrobial herbal extract. Examples of the antimicrobial herbal extract can be obtained by extracting one or more herbs selected from the group consisting of Rheum, Reynoutria, Coptis, Scutellaria and Phellodendron in a suitable liquid medium, as defined herein.

In another embodiment, the method further comprises mixing an anti-inflammatory herbal extract. Examples of the antimicrobial herbal extract can be obtained by extracting one or more herbs selected from the group consisting of Dendranthema, Ligustrum, Ilex, Patrinia, and Lithospermum (or Arnebia) in a suitable liquid medium, as defined herein.

In another embodiment, the method further comprises mixing a hemostatic herbal extract. Examples of the hemostatic herbal extract can be obtained by extracting one or more herbs selected from the group consisting of Panax, Rheum and Bletilla in a suitable liquid medium, as defined herein.

B. Burn and Other Wound Treatment

As noted herein, the herbal formulations of the present invention comprise active substances that non-invasively remove the necrotic tissues caused by a burn injury. The herbal formulations also comprise additional ingredients that impart pharmacological actions such as anti-microbial, hemostatic, pain-relieving, and anti-inflammatory functions. Moreover, the herbal formulations contain chemical or biological substances that can accelerate processes associated with wound healing, such as cell proliferation, migration and granulation.

In one aspect, the present invention provides a method of using an herbal formulation. The method may comprise applying to an affected area of a mammal (including a human) caused by burn, a skin lesion, or an ulcer, an effective amount of an herbal formulation comprising an aqueous extract of Paris and Sanguisorba.

In certain embodiments, the burn injury that the method of the present invention is useful in treating may be acute, meaning the time between the injury and treatment is less than 72 hours. In other embodiments, the burn injury that the method of the present invention is useful in treating may also have been chronically unhealed. Any burn injury or any other wound goes unhealed or shows no sign of healing for 72 hours or longer is considered “chronically unhealed” and the wound is considered a “chronic wound”.

Typically, the severity of burn injuries and chronic wounds varies greatly depending on the depth, area and location of the affected skin. Burn depth is generally categorized as first, second, or third-degree. First-degree burn typically results in minor injury of the outermost layer of skin. Second-degree burn involves the outermost skin as well as part of the skin underneath it. Third-degree burn destroys all the layers of skin, along with the blood vessels and nerves in the skin. In addition to the burn depth, the total area of the affected skin is also a significant indicator of the severity of burns. This is usually measured in terms of percentage of the total body surface area (BSA). The herbal formulations of the present invention are suitable for treating patients with first, second and third-degree burn injuries.

Healing at an acute wound site typically takes place in four stages. The initial stage is the “coagulation” stage where platelets appear at the wound area to stop bleeding and to agitate inflammatory cells. The second stage is the “inflammation” stage where neutrophils and macrophages provide antimicrobial functions and separate necrotic tissues from the viable. The third stage is the “migration/proliferation” stage where inflammatory cells such as macrophages continue to migrate into the wound space and fibroblasts start to accumulate. Macrophages play a critical role in angiogenesis, antimicrobial, debridement, cell recruitment and activation, and matrix synthesis regulation. Fibroblasts and collagen are the fundamental tissue for granulation. During this stage, skin cells migrate towards the wound center to epithelialize. The final stage is the “remodeling” stage when skin recovers and/or scar tissue forms. Each stage overlaps the previous one and each phase's timing can be expected. However, any factor, such as infection, nutrition deficiency, ischemia, dry wound environment, foreign bodies and anti-inflammatory therapy, can retard the healing process and cause the acute wounds to become “chronically unhealed”.

“An effective amount” of an herbal formulation refers to the amount of the herbal formulation that is effective in promoting one or more stages of the above-noted wound healing process. In other words, an effective amount of an herbal formulation is the amount that, when applied to an affected area, would promote processes such as the separation of necrotic tissue from healthy tissue, granulation, angiogenesis, skin cell growth and migration toward center, and/or epithelialization leading to closure of the wound surface at a statistically significant level. The effective amount of a particular herbal formulation may be determined by any appropriate method known in the art for measuring the effects of an agent or a composition of interest on various stages of the wound healing process. For example, an increasing amount of macrophages and/or amount of growth factors at a treated wound space compared to an untreated control provides a measure of the herbal formulation's effect on wound healing promotion of the “inflammation” and “migration/proliferation” stage; or the overall healing time for a treated wound compared to an untreated control wound provides a measure of the herbal formulation's effect on promoting wound healing.

For example, in certain embodiments, treatments need to be repeated daily during the first several days after the acute wound occurs, then to be repeated every other day in the later healing stages. For heavy necrotic tissue debridement, treatments need to be conducted as often as needed to thoroughly achieve a clean wound bed.

In one embodiment, the present invention provides a method comprising: applying to an affected area caused by burn, a skin lesion or an ulcer, an effective amount of an herbal formulation comprising an extract of Paris and Sanguisorba prepared by infusing Paris and Sanguisorba in a liquid medium having boiling point of no more than 160° C.

In one embodiment, the liquid medium is water. In another embodiment, the liquid medium is an alcohol. In another embodiment, the liquid medium is a mixture of water and an alcohol. In yet another embodiment, the liquid medium is acetone.

In another embodiment, the herbal formulation further comprises an antimicrobial herbal extract. For example, the antimicrobial herbal extract can be obtained by extracting Rheum, Reynoutria, Coptis, Scutellaria and Phellodendron or any combination thereof in a suitable liquid medium, as defined herein.

In another embodiment, the herbal formulation further comprises an anti-inflammatory herbal extract. For example, the antimicrobial herbal extract can be obtained by extracting Dendranthema, Ligustrum, Ilex, Patrinia, Lithospermum (or Arnebia) or a combination thereof in a suitable liquid medium, as defined herein.

In another embodiment, the herbal formulation further comprises a hemostatic herbal extract. For example, the hemostatic herbal extract can be obtained by extracting Panax, Bletilla, Rheum or a combination thereof in a suitable liquid medium, as defined herein.

In certain embodiments, the herbal formulation can be in the form of an aqueous solution, an ointment, a cream, a lotion or a gel.

The herbal formulations can be applied directly to a wound site caused by burns. Cleansing the wound site prior to the application is preferred but not necessary. The herbal formulation is particularly useful as an emergency first aid on a battlefield or a disaster site, where wound-cleansing and disinfection are not readily available.

In certain embodiments, the herbal formulations applied to wound site can be covered by a bandage. Bandages are particularly beneficial when aqueous-based formulations are used, because the bandages protect the wound site against external physical intrusion as well as keeping a moist environment to aid epithelization. Minor wounds, however, may not need to be covered by a bandage.

Typically, the removal of the herbal formulation (change of dressing) results in the removal of the necrotic tissue at the affected area. Thus, in another embodiment, the method further comprises removing necrotic tissue at the affected area by removing the herbal formulation. To alleviate any possible pain caused by dressing removal and to accelerate debridement, gauze covered with solid oil such as Vaseline or soaked with liquid oil such as vegetable oil can be placed between the wound and dressing bandage.

Advantageously, the non-invasive nature of the treatment prevents the growth of scar tissue. In addition, the treatment promotes rapid wound healing such that skin grafting is typically not necessary.

In certain embodiments, after the removal of the initial application of the herbal formulation, additional applications of the herbal formulation may be performed at the affected area, especially for large area wounds and deep wounds. For example, an appropriate amount of the herbal formulation may be applied 2-4 times a day during the first 2-3 days of the treatment to provide thorough debridement, wound cleansing and hemostasis functions, and then every other day during the later stages.

### EXAMPLES

### Example 1

### Exemplary Herbal Formulations and Method for Preparing Same

Exemplary Formulation:

Rhizoma of Paris Polyphylla Smith, 200 gram; Radix of Sanguisorba officinalis L. 200 gram; Radix et Rhizome of Rheum officinale Baill. 100 gram; Radix of Scutellaria baicalensis Georgi, 100 gram; Rhizoma of Polygonum cuspidatum Sieb. Et Zucc., 100 gram; Cortex of Phellodendron amurense Rupr. 100 gram, Rhizoma of Coptis chinensis Franch, 100 gram; Flos of Dendranthema morifolium (Ramat.) Tzvel, 100 gram and purified water 4200 gram.

Preparation:

Grind the above herbal substances into approximately 1×1 cm pieces and mix together, add 4000 gram of hot water, reflex boil for five hours and then cool to room temperature (25° C.). Separate the liquid phase from the solid residue. After the separation, the solid residue can be pressed for extra liquid extract. Filter the liquid phase to obtain a clear aqueous solution. Sterilize and pack the solution in closed containers.

### Example 2

### Topical Applications of the Herbal Formulation in Treating Burn Injuries

For a superficial second degree wounds, place gauze or medical cotton soaked with an herbal formulation directly over the wound area and wrap the wound area with a bandage. Care should be taken that the bandage is moist on the side contacting the gauze but remains dry on the outside. The dressing can be changed twice a day for the first 2-3 days and then every other day for the rest of the treatment. A superficial second degree wound can typically heal in less than 14 days. A thin layer of ointment such as Vaseline may be applied between the wound surface and the gauze to prevent pain caused by removing the dressing from the wound area during the dressing change and to facilitate debridement.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An herbal formulation comprising an extract of Paris and Sanguisorba prepared by infusing Paris and Sanguisorba in a liquid medium having a boiling point of no more than 160° C.

2. The herbal formulation of claim 1 wherein the liquid medium is water, an alcohol, acetone, or a combination thereof.

3. The herbal formulation of claim 1 further comprising an antimicrobial herbal extract.

4. The herbal formulation of claim 3 wherein the antimicrobial herbal extract is prepared by infusing Rheum, Reynoutria, Coptis, Scutellaria, Phellodendron or a combination thereof in the liquid medium.

5. The herbal formulation of claim 1 further comprising of an anti-inflammatory herbal extract.

6. The herbal formulation of claim 5 wherein the anti-inflammatory herbal extract is prepared by infusing Dendranthema, Ligustrum, Ilex, Patrinia, Lithospermum (or Arnebia) or a combination thereof in the liquid medium.

7. The herbal formulation of claim 1 further comprising of a hemostatic herbal extract.

8. The herbal formulation of claim 7 wherein the hemostatic herbal extract is prepared by infusing Bletilla, Panax, Rheum or a combination thereof in the liquid medium.

9. The herbal formulation of claim 1 further comprising a carrier.

10. The herbal formulation of claim 9 wherein the carrier is an oil.

11. The herbal formulation of claim 10 wherein the oil is almond oil, borage oil, canola oil, grape seed oil, jojoba oil, olive oil, soybean oil, sunflower oil, wheat germ oil, Vaseline, mineral oil, stearic acid, or mixtures thereof.

12. The herbal formulation of claim 10 further comprising a surfactant.

13. The herbal formulation of claim 9 wherein the carrier is a gel.

14. The herbal formulation of claim 13 wherein the gel is Carbomer, Xanthan Gum, Carrageenan, Acacia Gum, Guar Gum, Agar-Agar, alginates, carboxymethyl cellulose, hydroxyethyl cellulose and mixtures thereof.

15. The herbal formulation of claim 1 further comprising extracts prepared by infusing Rheum, Reynoutria, Coptis, Scutellaria, Phellodendron, and Dendranthema, Ligustrum and Ilex.

16. A method for preparing an herbal formulation, comprising:
a) infusing Paris and Sanguisorba in a liquid medium having a boiling point of no more than 160° C. to form a mixture;
(b) allowing the mixture to separate to a liquid phase and a solid phase; and
(c) separating the liquid phase from the solid phase, wherein the liquid phase comprises active substances from Paris and Sanguisorba.

17. The method of claim 16 further comprising:
(d) evaporating the liquid phase to remove all or part of the liquid medium to provide a concentrated extract.

18. The method of claim 16 wherein the liquid medium is water, an alcohol, acetone or a combination thereof.

19. The method of claim 16 wherein step (a) comprises infusing Paris, Sanguisorba, and one or more herbs selected from the group consisting of Rheum, Reynoutria, Coptis, Scutellaria, Phellodendron, Patrinia, Lithospermum (or Arnebia), Bletilla, Panax Dendranthema, Ligustrum, and Ilex.

20. The method of claim 16 further comprising: infusing the solid phase in fresh liquid medium and repeating steps (b) and (c).

21. The method of claim 17 further comprising mixing the concentrated extract with a carrier.

22. The method of claim 21 wherein the carrier is water, an oil, a cream or a gel.

23. A method of treating a mammal comprising: applying to an affected area caused by burn, a skin lesion or an ulcer, an effective amount of an herbal formulation comprising an aqueous extract of Paris and Sanguisorba.

24. The method of claim 23 further comprising removing necrotic tissues at the affected area by removing the herbal formulation.

25. The method of claim 23 further comprising forming a moist environment at the affected area.

26. The method of claim 23 wherein the herbal formulation further comprises an aqueous extract of one or more herbs selected from the group consisting of Rheum, Reynoutria, Coptis, Scutellaria, Phellodendron, Dendranthema, Ligustrum, Ilex, Patrinia, Bletilla, Panax and Lithospermum (or Arnebia).

27. A method of treating a mammal comprising:
(a) applying to an affected area caused by burn, a skin lesion or an ulcer, an effective amount of an herbal formulation prepared by a) infusing Paris and Sanguisorba in a liquid medium having boiling point of no more than 160° C. to form a mixture;
(b) allowing the mixture to separate to a liquid phase and a solid phase; and
(c) separating the liquid phase from the solid phase, wherein the liquid phase comprises active substances from Paris and Sanguisorba.

28. The method of claim 27 further comprising removing necrotic tissues at the affected area by removing the herbal formulation.

29. The method of claim 27 further comprising forming a moist environment at the affected area.

30. The method of claim 27 wherein step (a) comprises infusing Paris, Sanguisorba, and one or more herbs selected from the group consisting of Rheum, Reynoutria, Coptis, Scutellaria, Phellodendron, Dendranthema, Ligustrum, Ilex, Patrinia, Bletilla, Panax and Lithospermum (or Arnebia).

31. The method of claim 27 wherein the liquid medium is water, an alcohol, acetone or a combination thereof.
